(19) United States
(12) Patent Application Publication
(10) Pub. No.: US 2019/0183557 A1
(43) Pub. Date: Jun. 20, 2019

(54) COMPENSATION ASSEMBLY FOR FLUID INJECTION LINE OF CRYOGENIC BALLOON CATHETER SYSTEM

(71) Applicant: Cryterion Medical, Inc., Carlsbad, CA (US)

(72) Inventor: Eugene J. Jung, JR., San Diego, CA (US)

(21) Appl. No.: 16/223,353

(22) Filed: Dec. 18, 2018

Related U.S. Application Data
(60) Provisional application No. 62/607,863, filed on Dec. 19, 2017.

(51) Int. Cl. A61B 18/02; A61M 25/10

(52) U.S. Cl. CPC A61B 18/02; A61M 25/1018; A61B 2018/0022; A61M 25/1002; A61M 25/104

(57) ABSTRACT

A cryogenic balloon catheter system includes a cryoballoon, a guidewire lumen that extends through and is secured to the cryoballoon, an injection line receiver and a fluid injection line. The injection line receiver is fixedly secured to the guidewire lumen so that movement of the guidewire lumen moves the injection line receiver. The injection line receiver includes an interior chamber. The fluid injection line delivers cryogenic fluid to the interior chamber. In certain embodiments, the fluid injection line extends into the injection line receiver and allows relative movement between the fluid injection line and the injection line receiver. The fluid injection line is not affixed to the injection line receiver. The fluid injection line is not affixed to the guidewire lumen. The injection line receiver can include one or more injection line sealers that are positioned around the fluid injection line.

### CROSS-REFERENCE TO RELATED APPLICATION

This application claims priority to U.S. Provisional Application No. 62/607,863, filed on Dec. 19, 2017, and entitled “COMPENSATION ASSEMBLY FOR FLUID INJECTION LINE OF CRYOGENIC BALLOON CATHETER SYSTEM”. As far as permitted, the content of U.S. Provisional Application No. 62/607,863 is incorporated in its entirety herein by reference.

### TECHNICAL FIELD

The present disclosure relates to medical devices and methods for treating cardiac arrhythmias. More specifically, the disclosure relates to devices and methods for cardiac cryoablation.

### BACKGROUND

Cardiac arrhythmias, such as atrial fibrillation, involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. The energy delivery component of the system is typically at or near the most distal (farthest from the operator) portion of the catheter, and often at the tip of the device. Various forms of energy are used to ablate diseased heart tissue. These can include balloon cryotherapy which uses cryoballoons (also sometimes referred to herein as “balloon catheters”), ultrasound and laser energy, and radio frequency, to name a few. Atrial fibrillation is one of the most common arrhythmias treated using balloon cryotherapy. Atrial fibrillation is typically treated by pulmonary vein isolation, a procedure that removes unusual electrical conductivity in the pulmonary vein. In the earliest stages of the disease, paroxysmal atrial fibrillation, the treatment strategy involves isolating the pulmonary vein(s) from the left atrial chamber. Recently, the use of balloon cryotherapy procedures to treat atrial fibrillation has increased. In part, this stems from ease of use, shorter procedure times and improved patient outcomes.

In the case of balloon cryotherapy, one or more cryoballoons are maneuvered through the vascular system of the patient, and are ultimately positioned near or against targeted cardiac tissue. Once in position, the cryoballoons are inflated. Cryogenic fluid, such as liquid nitrous oxide, is delivered through a fluid injection line to an interior of the inflated cryoballoon(s) to cause tissue necrosis of the target cardiac tissue, which renders the tissue incapable of conducting electrical signals. Once the target tissue has been necrosed, the cryoballoons are then deflated and the balloon catheter is removed from the patient's body.

In many balloon catheters, the overall length of the balloon catheter can change during inflation and/or deflation. For example, while deflated, the cryoballoons are more elongated in length as the cryoballoons are stretched out. In these types of balloon catheters, during inflation and deflation of the cryoballoon(s), the overall length of the fluid injection line may need to be adjusted to maintain a distal end of the fluid injection line in proper position relative to the cryoballoon(s) during inflation and deflation. There is a continuing need for improved cryoablation balloon catheter designs.

### SUMMARY

The present disclosure is directed toward an injection line compensation assembly for a cryogenic balloon catheter system. The cryogenic balloon catheter system includes a cryoballoon and a guidewire lumen that extends through and is secured to the cryoballoon. In various embodiments, the injection line compensation assembly includes an injection line receiver and a fluid injection line. The injection line receiver is fixedly secured to the guidewire lumen so that movement of the guidewire lumen moves the injection line receiver. Further, the injection line receiver can include an interior chamber. The fluid injection line delivers a cryogenic fluid to the interior chamber of the injection line receiver. In certain embodiments, the fluid injection line extends into the injection line receiver and allows relative movement between the fluid injection line and the injection line receiver.

In various embodiments, the fluid injection line is not affixed to the injection line receiver.

In some embodiments, the fluid injection line is not affixed to the guidewire lumen.

In certain embodiments, the injection line receiver includes a plenum. In some such embodiments, the plenum substantially encircles a portion of the guidewire lumen.

In various embodiments, the injection line receiver includes a plurality of fluid ports that allow cryogenic fluid to exit the injection line receiver into the cryoballoon.

In some embodiments, the injection line receiver includes one or more injection line sealers that are positioned around a portion of the fluid injection line. In certain embodiments, the injection line sealer can include an O-ring. In various embodiments, the injection line sealer can be formed from a resilient material. In some embodiments, the injection line receiver can include a plurality of injection line sealers that are each positioned around a portion of the fluid injection line.

In certain embodiments, the injection line receiver is slidably movable relative to the fluid injection line.

In another embodiment, the injection line compensation assembly includes an injection line receiver and a fluid injection line. In certain embodiments, the injection line receiver is positioned within the handle assembly. Further, the injection line receiver can include an interior chamber. The fluid injection line can be at least partially positioned within the interior chamber of the injection line receiver. In various embodiments, the fluid injection line can be configured to receive a cryogenic fluid within the interior chamber. The fluid injection line can be fixed relative to at least a portion of the guidewire lumen so that movement of the guidewire lumen moves the fluid injection line relative to the injection line receiver.

In various embodiments, the fluid injection line can be movably secured to the injection line receiver. In certain embodiments, the injection line receiver can include one or more injection line sealers that are positioned around a portion of the fluid injection line.

In certain embodiments, the injection line sealer can include an O-ring. In various embodiments, the injection line sealer can be formed from a resilient material. In some embodiments, the injection line receiver can include a plurality of injection line sealers that are each positioned around a portion of the fluid injection line.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a simplified schematic side view illustration of a patient and an embodiment of a cryogenic balloon catheter system having features of the present disclosure;

FIG. 2 is a simplified side view of a portion of the patient and an embodiment of a portion of the cryogenic balloon catheter system including one embodiment of an injection line compensation assembly;

FIG. 3A is a partially cutaway side view of a portion of the cryogenic balloon catheter system, including a portion of a guidewire lumen and a portion of one embodiment of the injection line compensation assembly, shown in an extended position;

FIG. 3B is a partial cutaway side view of a portion of the guidewire lumen and a portion of the injection line compensation assembly illustrated in FIG. 3A, shown in a retracted position;

FIG. 4A is a perspective view of one embodiment of a portion of the injection line compensation assembly, including an injection line receiver;

FIG. 4B is a cross-sectional view of the injection line receiver taken on line 4B-4B in FIG. 4A; and

FIG. 5 is a partially cutaway perspective view of a portion of the cryogenic balloon catheter system, including a handle assembly and a portion of another embodiment of the injection line compensation assembly. While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure are described herein in the context of a balloon catheter steering assembly for a cryogenic balloon catheter system. Those of ordinary skill in the art will realize that the following detailed description of the present disclosure is illustrative only and is not intended to be in any way limiting. Other embodiments of the present disclosure will readily suggest themselves to such skilled persons having the benefit of this disclosure. Reference will now be made in detail to implementations of the present disclosure as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

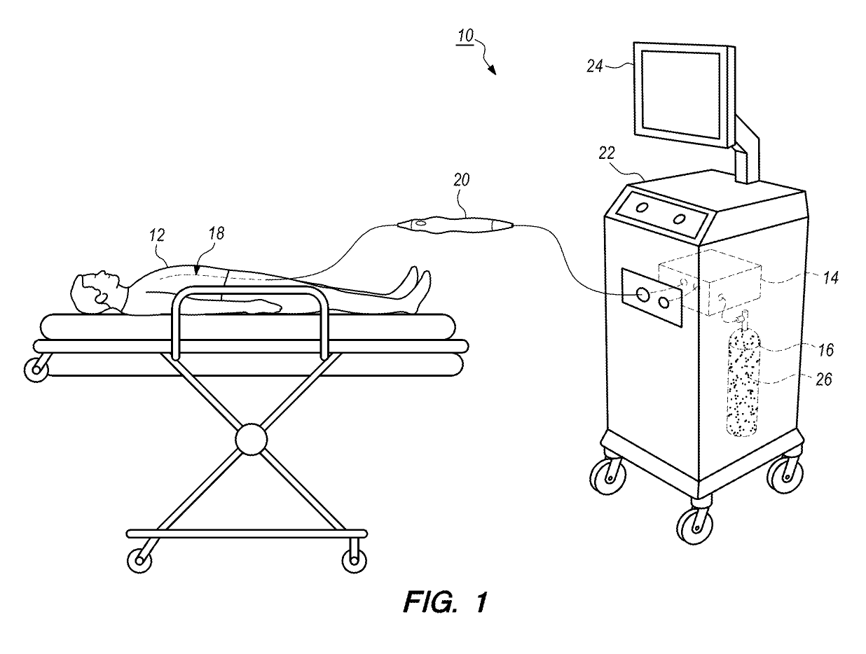

FIG. 1 is a schematic side view illustration of one embodiment of a cryogenic balloon catheter system 10 for use with a patient 12, which can be a human being or an animal. The design of the cryogenic balloon catheter system 10 can be varied. In certain embodiments such as the embodiment illustrated in FIG. 1, the cryogenic balloon catheter system 10 can include one or more of a control system 14, a fluid source 16, a balloon catheter 18, a handle assembly 20, a control console 22 and a graphical display 24. It is understood that although FIG. 1 illustrates the structures of the cryogenic balloon catheter system 10 in a particular position, sequence and/or order, these structures can be located in any suitably different position, sequence and/or order than that illustrated in FIG. 1. It is also understood that the cryogenic balloon catheter system 10 can include fewer or additional components than those specifically illustrated and described herein.

In various embodiments, the control system 14 can control release and/or retrieval of a cryogenic fluid 26 to and/or from the balloon catheter 18. In various embodiments, the control system 14 can control activation and/or deactivation of one or more other processes of the balloon catheter 18. Additionally, or in the alternative, the control system 14 can receive data and/or other information (hereinafter sometimes referred to as “sensor output”) from various structures within the cryogenic balloon catheter system 10. In some embodiments, the control system 14 can assimilate and/or integrate the sensor output, and/or any other data or information received from any structure within the cryogenic balloon catheter system 10. Additionally, or in the alternative, the control system 14 can control positioning of portions of the balloon catheter 18 within the body of the patient 12, and/or can control any other suitable functions of the balloon catheter 18.

The fluid source 16 contains the cryogenic fluid 26, which is delivered to the balloon catheter 18 with or without input from the control system 14 during a cryoablation procedure. The type of cryogenic fluid 26 that is used during the cryoablation procedure can vary. In one non-exclusive embodiment, the cryogenic fluid 26 can include liquid nitrous oxide. However, any other suitable cryogenic fluid 26 can be used.

The balloon catheter 18 is inserted into the body of the patient 12. In one embodiment, the balloon catheter 18 can be positioned within the body of the patient 12 using the control system 14. Alternatively, the balloon catheter 18 can be manually positioned within the body of the patient 12 by a health care professional (also sometimes referred to herein as an “operator”). In certain embodiments, the balloon catheter 18 is positioned within the body of the patient 12 utilizing the sensor output from the balloon catheter 18. In various embodiments, the sensor output is received by the control system 14, which then can provide the operator with information regarding the positioning of the balloon catheter 18. Based at least partially on the sensor output feedback received by the control system 14, the operator can adjust the positioning of the balloon catheter 18 within the body of the patient 12.

The handle assembly 20 is handled and used by the operator to operate, position and control the balloon catheter 18. The design and specific features of the handle assembly 20 can vary to suit the design requirements of the cryogenic balloon catheter system 10. In the embodiment illustrated in FIG. 1, the handle assembly 20 is separate from, but in electrical and/or fluid communication with the control system 14, the fluid source 16 and/or the graphical display 24. In some embodiments, the handle assembly 20 can integrate and/or include at least a portion of the control system 14 within an interior of the handle assembly 20. It is understood that the handle assembly 20 can include fewer or additional components than those specifically illustrated and described herein.

In the embodiment illustrated in FIG. 1, the control console 22 includes the control system 14, the fluid source 16 and the graphical display 24. However, in alternative embodiments, the control console 22 can contain additional structures not shown or described herein. Still alternatively, the control console 22 may not include various structures that are illustrated within the control console 22 in FIG. 1. For example, in one embodiment, the control console 22 does not include the graphical display 24.

The graphical display 24 provides the operator of the cryogenic balloon catheter system 10 with information that can be used before, during and after the cryoablation procedure. The specifics of the graphical display 24 can vary depending upon the design requirements of the cryogenic balloon catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time. Further, in various embodiments, the graphical display 24 can include one or more colors, different sizes, varying brightness, etc., that may act as alerts to the operator. Additionally, or in the alternative, the graphical display 24 can provide audio data or information to the operator.

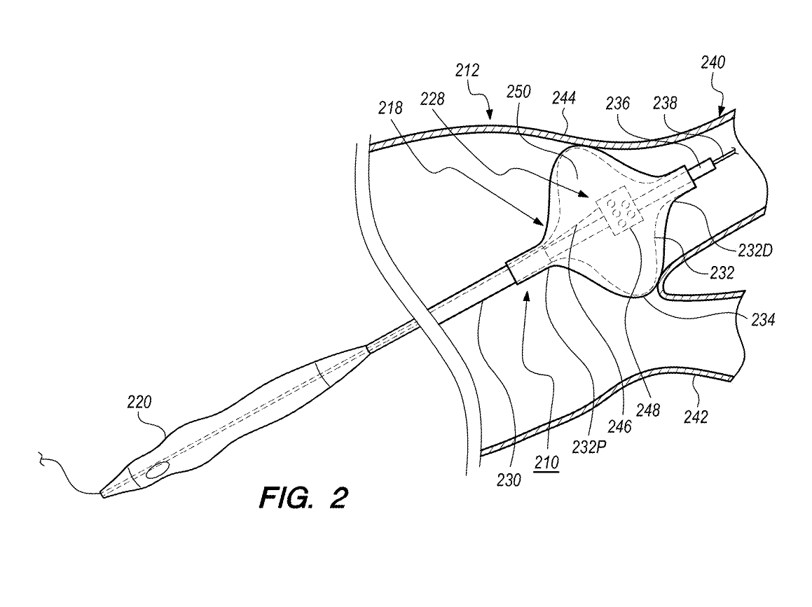

FIG. 2 is a simplified side view of a portion of the patient 212 and an embodiment of a portion of the cryogenic balloon catheter system 210. In this embodiment, the cryogenic balloon catheter system 210 includes a balloon catheter 218, a handle assembly 220 and an injection line compensation assembly 228 (also sometimes referred to herein as the “compensation assembly”).

The design of the balloon catheter 218 can be varied to suit the design requirements of the cryogenic balloon catheter assembly 210. In this embodiment, the balloon catheter 218 includes one or more of a catheter shaft 230, one or more balloons including at least one of an inner balloon 232 and an outer balloon 234, a guidewire lumen 236 and a guidewire 238. It is understood that the balloon catheter 218 can include other structures as well. However, for the sake of clarity, these other structures have been omitted from FIG. 2.

The catheter shaft 230 is positioned coaxially over the guidewire lumen 236. The design of the catheter shaft 230 can vary depending upon the design requirements of the balloon catheter 218. In one embodiment, a balloon proximal region 232P (nearer to the handle assembly 220) of the balloon(s) 232, 234 is secured to the catheter shaft 230, and a balloon distal region 232D (further from the handle assembly 220) of the balloon(s) 232, 234 is secured to the guidewire lumen 236.

The guidewire lumen 236 extends from the handle assembly 220 in a direction away from the handle assembly 220. The guidewire lumen can be mechanically (or otherwise) moved in a direction toward (retraction) or away (extension) from the handle assembly 220 to either extend or contract the balloon(s) 232, 234, since the distal region of the balloon(s) 232, 234, is secured to the guidewire lumen 236 and the proximal region of the balloon(s) is secured to the catheter shaft 230.

In the embodiment illustrated in FIG. 2, the balloon catheter 218 is positioned within the circulatory system 240 of the patient 212. The guidewire 238 and a portion of the guidewire lumen 236 are moved into a pulmonary vein 242 of the patient 212, and the catheter shaft 230 and the balloons 232, 234 are moved along the guidewire 238 to an ostium 244 of the pulmonary vein 242.

The design of the handle assembly 220 can vary. In the embodiment illustrated in FIG. 2, the handle assembly 220 can be used by an operator of the cryogenic balloon catheter system 210 to move portions of the balloon catheter 218. For example, the handle assembly 220 can include one or more steering mechanisms (not shown) that can be manipulated to steer the guidewire lumen 236 toward the desired pulmonary vein 242. The handle assembly 220 can have one or more additional or alternative suitable functions.

The injection line compensation assembly 228 compensates for changes in an overall length of the guidewire lumen 236 that extends away from the handle assembly 220 during extension and/or retraction of the guidewire lumen 236. In the embodiment illustrated in FIG. 2, the compensation assembly 228 includes a fluid injection line 246 and an injection line receiver 248.

The fluid injection line 246 delivers the cryogenic fluid 26 (illustrated in FIG. 1) to the cryoballoon(s) 232, 234, during a cryoablation procedure. The design of the fluid injection line 246 can vary. In one embodiment, the fluid injection line 246 is a tubular structure. In various embodiments, at least a portion of the fluid injection line 246 extends from the handle assembly 220, along or otherwise adjacent to the guidewire lumen 236, to a balloon interior 250. The fluid injection line 246 can be formed from any suitably flexible material. For example, in one non-exclusive embodiment, the fluid injection line 246 can be formed at least partially from nitinol.

In the embodiment illustrated in FIG. 2, the injection line receiver 248 movably receives a portion of the fluid injection line 246. The design of the injection line receiver 248 can vary. In one embodiment, the injection line receiver 248 can include a plenum or manifold-type of structure, as provided in greater detail herein. In the embodiment illustrated in FIG. 2, the injection line receiver 248 is affixed to, secured to, and moves in concert with, the guidewire lumen 236. In other words, as the guidewire lumen 236 extends and/or retracts during a cryoablation procedure, the injection line receiver 248 likewise moves with the guidewire lumen 236. Thus, the injection line receiver 248 can slidably move relative to the fluid injection line 246, which in various embodiments, is not secured to the guidewire lumen 236. Stated another way, the injection line receiver 248 can move forward and/or aft in a slidable manner over a portion of the fluid injection line 246. In certain embodiments, the cryogenic fluid 26 exits the fluid injection line 246 into the injection line receiver 248. The injection line receiver 248 can then distribute the cryogenic fluid 26 to the balloon interior 250 as needed during the cryoablation procedure, as provided in greater detail herein.

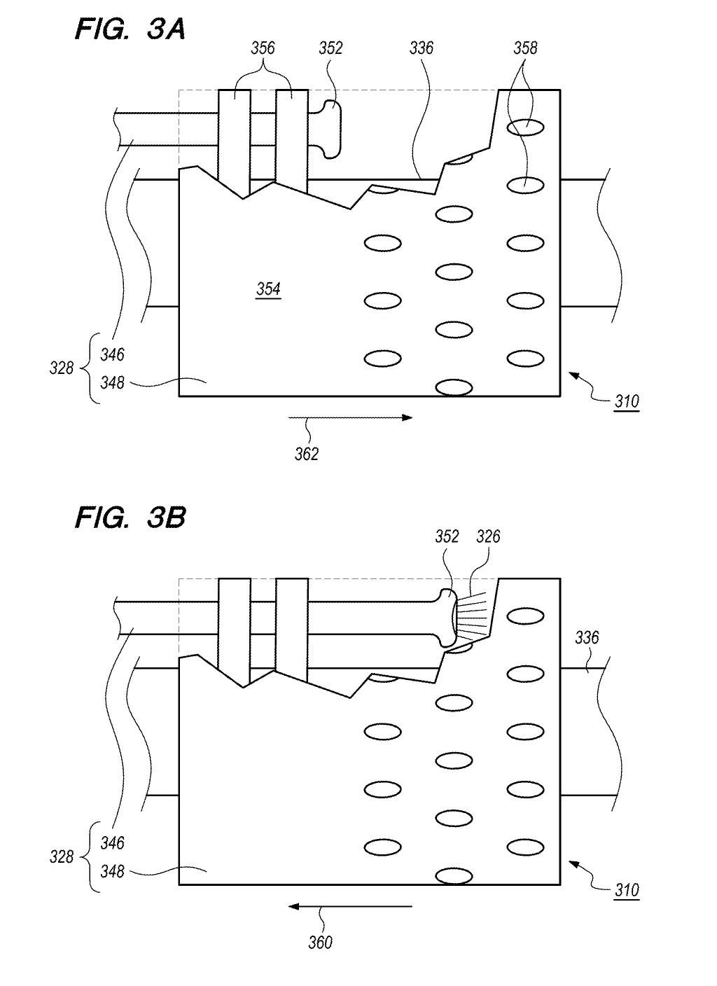

FIG. 3A is a partially cutaway side view of a portion of the cryogenic balloon catheter system 310 including a portion of a guidewire lumen 336 and a portion of one embodiment of the injection line compensation assembly 328, shown in an extended position. In this embodiment, the compensation assembly 328 includes the fluid injection line 346 and the injection line receiver 348.

In the extended position, the guidewire lumen 336 is extended distally away from the handle assembly 220 (illustrated in FIG. 2). In this position, at least a portion of the cryoballoon 232 (illustrated in FIG. 2) is likewise extended distally away from the handle assembly 220. Stated another way, the cryoballoon 232 is “stretched” lengthwise either prior to, or after ablation.

The fluid injection line 346 includes a line distal end 352 that is positioned within the injection line receiver 348. The line distal end 352 can have a wider or otherwise larger dimension than other portions of the fluid injection line 346, which maintains a positioning of the line distal end 352 within the injection line receiver 348, as provided in greater detail herein. Cryogenic fluid 326 (illustrated in FIG. 3B) can exit the fluid injection line 346 during inflation of the cryoballoon 232 and/or at other times during the cryoablation procedure.

In the embodiment illustrated in FIG. 3A, the injection line receiver 348 receives the line distal end 352 of the fluid injection line 346. The design of the injection line receiver 348, including the shape, size, materials used, and specific features, can vary. In the embodiment illustrated in FIG. 3A, the injection line receiver 348 can be a plenum or a manifold that receives and distributes the cryogenic fluid 326 to the balloon interior 250 (illustrated in FIG. 2). In this embodiment, the injection line receiver 348 is secured to the guidewire lumen 336, and is positioned to substantially encircle at least a portion of the guidewire lumen 336. With this design, the guidewire lumen 336 and the injection line receiver 348 move in unison relative to the fluid injection line 346. In one embodiment, the injection line receiver 348 can include one or more of a receiver body 354 and one or more injection line sealers 356 (two injection line sealers are illustrated in FIG. 3A).

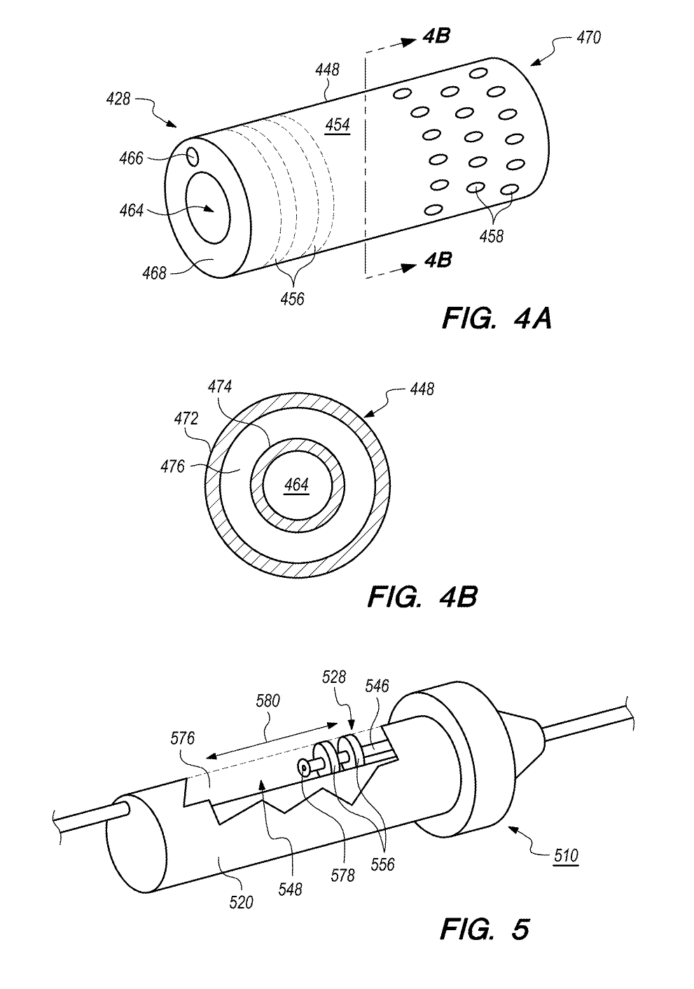

The receiver body 354 defines a mostly enclosed interior chamber 476 (illustrated in FIG. 4B) into which the cryogenic fluid 326 is delivered prior to being expelled into the balloon interior 250. In one embodiment, the receiver body 354 can be substantially cylindrical in shape. Alternatively, the receiver body 354 can be somewhat conical, frusto-conical, spherical, or bullet-shaped, as non-exclusive examples. Still alternatively, the receiver body 354 can have any other suitable shape, configuration or geometry. In the embodiment illustrated in FIG. 3A, the receiver body 354 includes one or more exit ports 358 through which the cryogenic fluid 326 exits the injection line receiver 348 into the cryoballoon 232. The exit ports 358 can be arranged in any suitable pattern to increase the likelihood of even distribution of the cryogenic fluid 326 in a desired portion of the cryoballoon 232. Further, the shape of each exit port 358 can be tailored to increase the likelihood of even distribution of the cryogenic fluid 326 in a desired portion of the cryoballoon 232.

The injection line sealer(s) 356 form a seal around the fluid injection line 346 and within a portion of the injection line receiver 348 to inhibit cryogenic fluid 326 from exiting the receiver body 354 through any avenue other than the exit ports 358. Additionally, or in the alternative, the injection line sealer 356 inhibits the fluid injection line 346 from being completely removed from the injection line receiver 348, given the enlarged line distal end 352. In the embodiment illustrated in FIG. 3A, the injection line sealers 356 can be annular or somewhat washer shaped. Alternatively, the injection line sealers 356 can have any other suitable shape. In one embodiment, the injection line sealers 356 can be formed from a resilient material such as rubber, plastic, or the like.

FIG. 3B is a partially cutaway side view of a portion of the cryogenic balloon catheter system 310 including a portion of a guidewire lumen 336 and a portion of one embodiment of the injection line compensation assembly 328, shown in a retracted position.

In the retracted position, the guidewire lumen 336 is retracted toward the handle assembly 220 (illustrated in FIG. 2). In this position, at least a portion of the cryoballoon 232 (illustrated in FIG. 2) is likewise retracted toward the handle assembly 220 in preparation for ablation. Stated another way, the cryoballoon 232 is contracted lengthwise in preparation for and/or during ablation.

In this embodiment, the guidewire lumen 336 and the injection line receiver 348 move substantially in unison relative to the fluid injection line 346. To move from the extended position illustrated in FIG. 3A to the retracted position illustrated in FIG. 3B, the guidewire lumen 336 is pulled or otherwise moved in a direction indicated by arrow 360. Because the injection line receiver 348 is secured or otherwise connected to the guidewire lumen 336, moving the guidewire lumen 336 also simultaneously moves the injection line receiver 348 in a direction 360, for example.

Conversely, to move from the retracted position illustrated in FIG. 3B to the extended position illustrated in FIG. 3A, the guidewire lumen 336 is pushed or otherwise moved in a direction indicated by arrow 362 (illustrated in FIG. 3A). Because the injection line receiver 348 is secured or otherwise connected to the guidewire lumen 336, moving the guidewire lumen 336 also simultaneously moves the injection line receiver 348 in a direction 362, for example.

FIG. 4A is a perspective view of one embodiment of a portion of the injection line compensation assembly 428, including one embodiment of the injection line receiver 448. The fluid injection line 346 has been omitted from FIG. 4A for clarity. In this embodiment, the injection line receiver 448 includes a receiver body 454, one or more injection line sealers 456 (illustrated in phantom in FIG. 4A), one or more exit ports 458, a guidewire lumen receiver 464, an injection line aperture 466, a body proximal end 468 and a body distal end 470. The receiver body 454 can have a configuration that is somewhat similar to that previously described herein. Alternatively, the receiver body 454 can have a different configuration. In this embodiment, the exit ports 458 are positioned at a plurality of locations around a circumference of the receiver body 454.

The guidewire lumen receiver 464 receives the guidewire lumen 336 (illustrated in FIG. 3A, for example). The injection line receiver 448 can be secured to the guidewire lumen 336 by any suitable manner. For example, the injection line receiver 448 can be adhered to the guidewire lumen 336 with an adhesive material. Alternatively, the injection line receiver 448 can be welded to the guidewire lumen 336, as one non-exclusive embodiment.

The injection line aperture 466 allows access or insertion of the fluid injection line 346 (illustrated in FIG. 3A) into an interior chamber 476 (illustrated in FIG. 4B) of the injection line receiver 448.

FIG. 4B is a cross-sectional view of the injection line receiver 448 taken on line 4B-4B in FIG. 4A. In this embodiment, the injection line receiver 448 includes an outer wall 472, an inner wall 474 and an interior chamber 476 that is defined by the space formed between the outer wall 472 and the inner wall 474. It is understood that the configuration of the outer wall 472, the inner wall 474 and the interior chamber 476 can be varied from that illustrated in FIG. 4B.

FIG. 5 is a partially cutaway perspective view of a portion of an embodiment of the cryogenic balloon catheter system 510, including a handle assembly 520 and a portion of another embodiment of the injection line compensation assembly 528. The design of the handle assembly 520 can vary to suit the design requirements of the cryogenic balloon catheter system 510. In this embodiment, at least a portion of the injection line compensation assembly 528 can be positioned substantially within the handle assembly 520.

For example, in the embodiment illustrated in FIG. 5, the compensation assembly 528 includes the fluid injection line 546 and an injection line receiver 548. In one embodiment, the fluid injection line 546 can include a line distal end (not shown in FIG. 5) and a line proximal end 578. The line distal end extends into the cryoballoon interior 250 (illustrated in FIG. 2, for example). However, in this embodiment, the line distal end of the fluid injection line 546 is secured, coupled or otherwise attached to the guidewire lumen 336 (illustrated in FIG. 3A, for example). With this design, the fluid injection line 546 moves in concert with the guidewire lumen 336.

The line proximal end 578 is positioned within the injection line receiver 548 in the handle assembly 520. Because the fluid injection line 546 moves in concert with the guidewire lumen 336, the line proximal end 578 moves within the injection line receiver 548 in both a forward and an aft direction shown by arrow 580.

In this embodiment, the injection line receiver 548 can include one or more injection line sealers 556 (two injection line sealers 556 are illustrated in FIG. 5) that seal an interior chamber 576 to allow a more consistent flow of cryogenic fluid 26 (illustrated in FIG. 1) to the line proximal end 578 while inhibiting leakage of cryogenic fluid 26 to unwanted regions of the cryogenic balloon catheter system 510.

It is understood that although a number of different embodiments of the cryogenic balloon catheter system and the injection line compensation assembly have been illustrated and described herein, one or more features of any one embodiment can be combined with one or more features of one or more of the other embodiments, provided that such combination satisfies the intent of the present disclosure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A cryoablation catheter comprising:
a catheter shaft;
a guidewire lumen disposed within and slidable relative to the shaft;
a cryoballoon having a first end portion secured to a distal end of the shaft, and a second end portion attached to the guidewire lumen distal to the distal end of the shaft;
an injection line receiver that is fixedly secured to the guidewire lumen so that movement of the guidewire lumen moves the injection line receiver, the injection line receiver including an interior chamber; and
a fluid injection line that delivers a cryogenic fluid to the interior chamber of the injection line receiver, the fluid injection line extending into the injection line receiver and allowing relative movement between the fluid injection line and the injection line receiver.

2. The cryoablation catheter of claim 1, wherein the fluid injection line is not affixed to the injection line receiver.

3. The cryoablation catheter of claim 1, wherein the fluid injection line is not affixed to the guidewire lumen.

4. The cryoablation catheter of claim 1, wherein the injection line receiver includes a plenum including a plurality of fluid ports.

5. The cryoablation catheter of claim 4, wherein the plenum encircles a portion of the guidewire lumen.

6. The cryoablation catheter of claim 1, wherein the injection line receiver includes a plurality of fluid ports that allow cryogenic fluid to exit the injection line receiver into the cryoballoon.

7. The cryoablation catheter of claim 1, wherein the injection line receiver includes an injection line sealer positioned around a portion of the fluid injection line.

8. The cryoablation catheter of claim 7, wherein the injection line sealer includes an O-ring.

9. The cryoablation catheter of claim 7, wherein the injection line sealer is formed from a resilient material.

10. The cryoablation catheter of claim 1, wherein the injection line receiver includes a plurality of injection line sealers that are each positioned around a portion of the fluid injection line.

11. The cryoablation catheter of claim 1, wherein the injection line receiver is slidably movable relative to the fluid injection line.

12. A cryoablation catheter comprising:
a handle assembly;
a cryoballoon;
a guidewire lumen that extends through and is secured to the cryoballoon;
an injection line receiver positioned within the handle assembly, the injection line receiver including an interior chamber; and
a fluid injection line that is at least partially positioned within the interior chamber of the injection line receiver, the fluid injection line being configured to receive a cryogenic fluid within the interior chamber, the fluid injection line being fixed relative to at least a portion of the guidewire lumen so that movement of the guidewire lumen moves the fluid injection line relative to the injection line receiver.

13. The cryoablation catheter of claim 12, wherein the fluid injection line is movably secured to the injection line receiver.

14. The cryoablation catheter of claim 12, wherein the injection line receiver includes an injection line sealer positioned around a portion of the fluid injection line.

15. The cryoablation catheter of claim 14, wherein the injection line sealer includes an O-ring.

16. The cryoablation catheter of claim 14, wherein the injection line sealer is formed from a resilient material.

17. The cryoablation catheter of claim 12, wherein the injection line receiver includes a plurality of injection line sealers that are each positioned around a portion of the fluid injection line.

18. The cryoablation catheter of claim 12, wherein the injection line receiver is slidably movable relative to the fluid injection line.

19. A cryogenic fluid injection assembly for a cryoablation catheter having a handle assembly, a catheter shaft extending from the handle assembly, a guidewire lumen slidably extending within the catheter shaft, and a cryoballoon at a distal end of the catheter shaft and attached to the guidewire lumen, the cryogenic fluid injection assembly comprising:
an injection line receiver secured to the guidewire lumen so that movement of the guidewire lumen moves the injection line receiver, the injection line receiver including an interior chamber; and
a fluid injection line that delivers a cryogenic fluid to the interior chamber of the injection line receiver, the fluid injection line extending into the injection line receiver and allowing relative movement between the fluid injection line and the injection line receiver.

20. The cryogenic fluid injection assembly of claim 19, wherein the injection line receiver includes a plurality of fluid ports that allow cryogenic fluid to exit the injection line receiver into the cryoballoon.
